(19) United States
(12) Patent Application Publication
(10) Pub. No.: US 2007/0042337 A1
(43) Pub. Date: Feb. 22, 2007

(54) Isochoric method and device for reducing the probability of ice nucleation during preservation of biological matter at subzero centigrade temperatures

(75) Inventors: Boris Rubinsky, Albany, CA (US); Stephanie Szobota, Albany, CA (US)

Correspondence Address: GORDON & REES LLP, 101 WEST BROADWAY, SUITE 1600, SAN DIEGO, CA, 92101, US

(73) Assignee: The Regents of the University of California, Berkeley, CA (US)

(21) Appl. No.: 11/485,922

(22) Filed: Jul. 12, 2006

Related U.S. Application Data
(60) Provisional application No. 60/701,236, filed on Jul. 20, 2005.

(51) Int. Cl. A61L 2/02; A01N 1/02

(52) U.S. Cl. 435/1.1; 422/1; 435/2

(57) ABSTRACT

Because ice-I is less dense than water, the formation of an ice nucleus in an isochoric (constant volume) system containing water at pressures lower than about 200 MPa will cause an increase in pressure. This increase in pressure increases the energy required for reducing the probability for ice nucleation in an isochoric system containing water. In the present invention, a system for decreasing the probability of ice nucleation in a system containing water based on isochoric cooling and warming is provided. Reduction in the probability of ice nucleation has use in biological material preservation at low temperatures in: a supercooled state, by rapid freezing and through vitrification.

### RELATED APPLICATION

The present application claims priority under 35 U.S.C. Section 119 to U.S. Provisional patent application 60/701,236, entitled “Method and Device for Cryopreservation In Water In A Liquid State At Subzero Celsius Temperatures In A Supercooled Form”, filed Jul. 20, 2005.

### TECHNICAL FIELD

The present invention is related to methods and devices for reducing the probability of ice nucleation during cryopreservation of biological matter.

### BACKGROUND OF THE INVENTION

(a) Overview of Nucleation Theory

(i) Phase Transition in Thermodynamic Equilibrium

When water and ice are together in a solution, the temperature is fixed and determined from thermodynamic equilibrium as a function of pressure and water solution composition. This equilibrium temperature is often referred to as the melting point of ice or the freezing point of water. At atmospheric pressure, in pure water the temperature will adjust to 0° C., as long as both phases are present. Water in a liquid form at a temperature below the thermodynamic equilibrium temperature for phase transformation is known as “supercooled” and is considered in a thermodynamically metastable state. While the thermodynamic conditions of equilibrium are fixed the process of freezing and thawing requires excursions in the metastable state. In fact, water must be subcooled to below the equilibrium thermodynamic phase transition temperature in order to freeze, and ice must be warmed slightly above the phase transition temperature in order to melt. Melting, however, begins immediately once the temperature exceeds the phase transition temperature, no matter how slight the margin, whereas freezing may not occur until the water is subcooled to several degrees below the equilibrium temperature. The difference between these processes is because the transformation of water into ice must be initiated by a microscopic ice cluster, called a “nucleus”.

(ii) Ice Nucleation

The dynamic process of phase transformation relates to the formation of this “nucleus” and is a probabilistic event. Combinations of molecules with the molecular structure of ice continuously and randomly form and disassemble in the fluid as a result of the random motion of water molecules and microscale fluctuations in water temperature and density. If an ice nucleus larger than the critical size randomly assembles from water molecules in the subcooled water, ice will spontaneously propagate and freezing begins (Franks, F., Ed. (1982). Water: a comprehensive treatise. New York, Plenum Press.), and (Hobbs P V (1974). Ice physics. Oxford, Clarendon Press). This is called homogeneous nucleation. Homogeneous nucleation is more likely to occur in large volumes of water and at very low temperatures. (Given a larger number of water molecules, there is a greater probability of several molecules randomly assembling into a critical cluster.) Experiments have shown that water under atmospheric, isobaric conditions can be subcooled to about −45° C. before homogeneous nucleation occurs (Ford, I., J. (2001). “Properties of ice clusters from an analysis of freezing nucleation.” J. Phys. Chem. B 105: 11649-11655.) For this reason, −45° C. has been labeled the homogeneous nucleation temperature of water. Such experiments require a micro-sized droplet of water to minimize the probability that a critical cluster will randomly assemble. The homogeneous nucleation temperature corresponds to a critical cluster of about 25 molecules (a radius of 4 angstroms).

Heterogeneous nucleation occurs when water molecules assemble on the surface of an impurity with a contact angle which allows the water molecules to form a portion of the critical-sized sphere. The impurity takes up much of the volume that would have been required by a critical-sized cluster, and as a result, only a fraction of the water molecules needed for homogeneous nucleation are actually required. Smaller contact angles require fewer molecules to achieve the critical radius. The contact angle between water and bulk ice is 0, so introducing a piece of ice into subcooled water triggers immediate ice propagation. Water forms a large contact angle with hydrophobic surfaces, and consequently, heterogeneous nucleation on a hydrophobic surface requires nearly as many molecules as homogeneous nucleation. Impurities that cause heterogeneous nucleation are sometimes called nucleators. Heterogeneous nucleation can also occur on the interior surfaces of a vessel that contains subcooled water.

An example of heterogeneous nucleation of ice is illustrated in FIG. 1.

(b) Overview of Cryopreservation:

The ability to preserve biological materials for an extended period of time is of great importance to fields like medicine, agriculture, food industry and biotechnology. The preservation of organs, tissues, cells or biological molecules requires that chemical reactions in which they are involved are slowed or halted during preservation and then restored. The biochemical reactions, known in living biological matter collectively as metabolism, can be slowed by lowering the temperature, as is generally the case with all chemical reactions. Preservation is considered successful when the biological material functions normally when restored to physiological temperatures. However, the temperature excursion from physiological conditions to sub-physiological conditions and back involves a large variety of mechanisms of damage. Overcoming these modes of damage is the goal of the field of cryopreservation.

Ideally, a biological material would be stored for preservation at absolute zero, the temperature at which all activity ceases. Because organic molecules, cells and organisms exist in solutions of water, cooling below the physiological temperatures has two temperature regimes related to the eventual phase transition of water into ice: (a) temperatures above the thermodynamic equilibrium of ice and solution and temperatures below the thermodynamic equilibrium of ice and water. Low temperature preservation is divided into three categories: (a) hypothermic preservation, at temperatures above the thermodynamic equilibrium phase transition temperature; (b) freezing preservation at temperatures below the thermodynamic equilibrium phase transition temperature in the presence of ice; and (c) supercooling preservation in which the aqueous solution does not freeze at all and remains in a liquid state to cryogenic temperatures either because it takes a high viscosity liquid glass state (vitrification) or because it exists in a metastable state of thermodynamic supercooling. A comprehensive literature review on the mechanisms of damage to biological materials during these three modes of preservation can be found in (Rubinsky, B. (2000). Cryosurgery. Annual Review of Biomedical Engineering. M. L. Yarmush, K. R. Diller and M. Toner. 2: 157-187.) and (Rubinsky, B. (2002). Low temperature preservation of biological organs and tissues. Future Strategies for tissue and organ replacement. J. Polak, L. Hench and P. Kemp. London, GB, Imperial Press: 27-49.) and (Rubinsky, B. (2003). “Principles of low temperature cell preservation.” Heart failure reviews 8(3): 277-285.)

Preservation by hypothermia is characterized by a sub-physiological temperature, a state of thermodynamic equilibrium and the absence of ice crystallization. The cell membrane, which consists of a lipid bilayer and integrated proteins, maintains a fluid-like state at physiological temperatures. At sub-physiological temperatures, the lipid bilayer transitions into a gel (Morris, G. J. and A. Clarke, Eds. (1981). The effects of low temperature on biological membranes. London, Academic Press.) This lipid-phase transition causes leakiness in the cell membrane and the aggregation of membrane-bound proteins. The flux of ions across the cell membrane is no longer controlled, and ionic imbalances can denature intracellular proteins and cause swelling that is detrimental to the cell. The cytoskeleton, which partly relies on its bonds formed with the cell membrane, is also susceptible to damage (Grout, B., W., W., and G. J. Morris, Eds. (1987). The effect of low temperature on biological systems. London, Edward Arnold Ltd.). Besides the cell membrane, any other membranous structure in the cell can be compromised by a lipid-phase transition. Certain cell types, such as platelets, have greater survival at only modest hypothermic temperatures, because the benefit of reduced metabolism (increased ischemic tolerance resulting from a reduction in oxygen demand) is outweighed by the harm of uncontrolled ion flux. The stresses induced by low temperatures have also been shown to trigger apoptosis (self-regulated cell death) (Baust, J., M., R. Van Buskirk, et al. (2000). “Cell viability improves following inhibition of cryopreservation-induced apoptosis.” In Vitro Cellular & Developmental Biology. Animal. 36(4): 262-270.) Al these modes of damage could be avoided by cooling to lower temperatures, below the equilibrium phase transition temperature of ice. However the sub-freezing temperatures induce additional mechanisms of damage related to the formation of ice.

The temperatures associated with freezing preservation further reduce metabolism; however, freezing preservation is subject to damage caused by ice crystallization. The mechanisms of damage relate to the cooling rates during freezing. In the cooling rate regime known as, slow cooling (Mazur, P. (1970). “Cryobiology: the freezing of biological systems.” Science 68: 939-949), ice crystallization will first occur in larger fluid volumes, such as the storage solution surrounding the biological material, in the vasculature, and in the interstitial space (Ishiguro, H. and B. Rubinsky (1994). “Mechanical interactions between ice crystals and red blood cells during directional solidification.” Cryobiology 31: 483-500) Mechanical damage results when expanding ice crystals puncture or crush nearby cells. The freezing also triggers a cascade of events leading to chemical damage. When a solution begins to freeze, the concentration of solutes in the unfrozen fluid increases, because the crystalline structure of ice is very tight and cannot incorporate impurities or solutes. The hypertonic extracellular solution causes an osmotic gradient that drives water from the intracellular space. As a consequence, the intracellular solution becomes hypertonic, which can cause irreversible chemical damage to the cell (Lovelock, J., E., (1953). “The haemolysis of human red blood cells by freezing and thawing.” Biochem, Biophys. Acta 10: 412-426), (Mazur, supra), (Tasutani and Rubinsky, supra). The osmotic cascade brought on by freezing can be interrupted with cooling rates that reduce the temperature of the biological substance faster than water can exit cells by osmosis (Mazur, supra), (Merryman, H., T. (1966). Cryobiology. New York, Academic Press). A plot of cell survival as a function of cooling rate has an inverse-U shape, with survival increasing up to an optimal cooling rate and then decreasing at higher rates. These higher cooling rates allow the intracellular fluid to reach lower temperatures in a supercooled state, and experiments have correlated the decrease in cell survival with the sudden formation of intracellular ice in the supercooled fluid (Diller, K., R., and E. Cravalho, G. (1970). “A cryomicroscope for the study of freezing and thawing processes in biological cells.” Cryobiology 7: 191-199.), (Mazur, supra), (Tasutani and Rubinsky, supra). Intracellular ice formation is almost always lethal to cells (Mazur, supra). The intracellular ice formation is directly related to the homogeneous or heterogeneous nucleation discussed earlier.

Cryopreservation by freezing is currently the main method that is partially successful for the long term preservation of biological materials. Many of the damage mechanisms described above for freezing can be mitigated through the use of chemical additives, controlled cooling/rewarming rates, and pressure. Chemical additives, or cryoprotectants, have been shown to control intracellular and extracellular ionic concentrations and prevent osmotic cell damage (Polge, S., A. Smith, V., et al. (1948). “Revival of spermatozoa after vitrification and dehydration at low temperature.” Nature 164: 666.). A pioneering study by Audrey Smith in 1957 demonstrated that hamster hearts resumed rhythmic beating after perfusion with 15% glycerol and exposure to −20° C. (Smith, A. U. (1961). The effects of glycerol and of freezing on mammalian organs. Biological Effects of Freezing and Supercooling. A. U. Smith. London, Edward Arnold.). Glycerol, ethylene glycol, and dimethyl sulfoxide (DMSO) penetrate the cell membrane and depress the freezing temperature of the intracellular solution. Unfortunately, cryoprotectants tend to be most effective at high concentrations which are biologically toxic, and cryoprotectant concentration increases even further during the solute-rejection that occurs with freezing. Out of convenience, most cryopreservation protocols take place under isobaric (constant pressure) conditions at a pressure of 1 atm. Hyperbaric pressure, however, can prevent ice formation at low temperatures, although the elevated stress can be lethal to living cells (Fahy, G. M., D. R. MacFarlane, et al. (1984). “Vitrification as an approach to cryopreservation.” Cryobiology 21: 407-427.), (Suppes, G. J., S. Egan, et al. (2003). “Impact of high pressure freezing on DH5a Eschericia coli and red blood cells.” Cryobiology 47: 93-101.), and Takahashi, T., K. Kakita, et al. (2000). “Functional integrity of the rat liver after subzero preservation under high pressure. High Pressure.” Transplant. Proc. 32: 1634-6.) Recently, it has been found through a thermodynamic analysis that freezing under isochoric conditions, i.e. in a constant volume system, reduces the hazards of both cryoprotectant concentration and hyperbaric pressures and could improve the outcome of a cryopreservation protocol that involves freezing (Rubinsky, B., A. P. Perez, et al. (2005). “The thermodynamic principles of isochoric cryopreservation.” Cryobiology 50: 121-138.). However, the finding reported in (Rubinsky, Perez, supra) deals with situations in which there is ice in the system.

Ice formation during freezing is the primary factor related to damage during cryopreservation at cryogenic temperatures. Luyet was the first to show that the damage due to ice formation during cryopreservation could be avoided by cooling to cryogenic temperatures without ice formation, in a process known as vitrification. Vitrification (also known as glass-transition) occurs when a fluid is cooled until it becomes sufficiently viscous that the fluid motion of the molecules is halted. The molecules are locked into a solid-like state but keep a disordered (non-crystalline, liquid) arrangement. For pure water at atmospheric pressure, vitrification corresponds to a temperature of about −138° C. (Tg, the glass-transition temperature of water) (Franks, supra). Vitrifying a biological substance would prevent a majority of the cell damage that is normally encountered during cryopreservation. Biological preservation by vitrification, would reduce metabolic rates while preventing the damage associated with ice crystallization and could allow storage of biological materials indefinitely.

To achieve vitrification, the formation of the critical nucleus, discussed in the previous section, needs to be avoided. The goal of cryopreservation protocols with vitrification is to reduce the probability of ice crystal nucleation and formation during cooling to cryogenic temperatures and during re-warming to physiological temperatures. To this end currently, cryopreservation protocols targeting vitrification utilize hyperbaric pressure, chemical agents, high concentrations of cryoprotectants (which are often toxic themselves) and fast cooling and warming rates to minimize or prevent ice crystallization during the excursion to and from vitrification temperatures. Each of these techniques presents biological hazards, such as crushing damage from high pressure, chemical toxicity, osmotic lysis and cold shock. Currently vitrification is performed in an isobaric (constant pressure) system with high concentrations of additives (Fahy G M, W. B., Wu J, Phan J, Rasch C, Chang A, Zendejas E (2004). “Cryopreservation of organs by vitrification: perspectives and recent advances.” Cryobiology 48: 157-178.)

To date, preservation of biological substances has been moderately successful at best and mostly applies to some types of cells. Organ and tissue transplantation, as practiced today, relies on preservation by hypothermia. Organ preservation by freezing or vitrification, which has not yet been achieved, could allow storage of biological materials indefinitely. Low-temperature preservation can also be applied to in vitro fertilization, food storage, and other areas. Optimizing the use of cryoprotectants, pressure, and cooling/rewarming rates in order to improve biological survival and technological feasibility continues to be a central area of research, as is developing a better understanding of physics and material behavior at low temperatures.

### SUMMARY OF THE INVENTION

The present invention provides a method of cryopreservation of a biological sample, by: placing a biological sample in a fluid in a chamber; and supercooling the fluid in the chamber under isochoric conditions, without actively inducing ice nucleation in the fluid, thereby reducing the probability of ice nucleation in the fluid, and thereby cryopreserving the biological sample. The fluid may optionally be pure water or an aqueous solution with organic molecules therein. The biological sample may optionally be a cell, a group of cells, an organ and an organism.

In optional aspects, a compound with cryoprotective properties, or properties that promote vitrification may be added to the fluid, or to the biological sample. Such compounds may include glycerol, ethylene glycol, and DMSO (dimethyl sulfoxide). In other optional aspects, a chemical that inhibits nucleation may be added to the fluid. Such chemical may include antifreeze proteins and oily hydrocarbons.

In preferred aspects, the fluid may be supercooled to temperatures in the range of from 0 C to −273.25 C. The temperature may then be kept constant, thereby cryopreserving the biological sample. Later, the biological material may be warmed for use by increasing the above the temperature of the formation of ice (i.e.: above 0 degrees).

In preferred aspects, the fluid may be supercooled by immersing the fluid chamber in an exterior fluid bath.

To further reduce the probability of ice nucleation, the fluid chamber preferably does not contain ice nucleating agents, gases, or materials that absorb gases. Optionally as well, the fluid chamber contains agents that inhibit nucleation, including, but not limited to, antifreeze proteins and thermal histeresys proteins.

The present invention can be used with pure water or an aqueous solution in a liquid like state during cooling to maintenance at and warming from subzero Celsius temperatures. Specifically, the present invention provides a system for reducing the probability of ice nucleation and formation thereby facilitating the retention of water, pure or in a solution, in a liquid form to cryogenic temperatures. In preferred aspects, this is accomplished by keeping the fluid in the system as close to isochoric (constant volume) conditions as possible.

The present inventors have analyzed the thermodynamics of ice nucleation under isochoric conditions. In accordance with the present invention, a system is provided to maintain water or aqueous solutions in an isochoric, (constant volume) system that reduces the probability of ice nucleation and formation.

The present invention is ideally suited for application with cryopreservation. In accordance with the present invention, a system of cryopreservation in an isochoric chamber is provided. As will be shown, the present system advantageously leads to a reduction in the probability for nucleation in water and aqueous solutions. Therefore, the present invention has applications in: (a) preservation of biological materials in a liquid form in a thermodynamically supercooled state, (b) inhibition of ice formation inside cells (the system is the space inside cells) during rapid cooling and (c) vitrification. The present system advantageously leads to vitrification and eliminates or reduces the need for elevated pressures, high concentrations of chemical additives and high cooling and warming rates in the current techniques.

The present invention is thus particularly useful for preserving biological materials such as: solutions of biological compounds, cell components, cells, tissues organs, and organisms at subzero centigrade temperatures. Reducing the temperature has the effect of reducing the rate of chemical reactions in these biological materials, and thus the present system can be used for the preservation of these biological materials. In preferred aspects, the temperature in the system is reduced from zero centigrade to absolute zero (−273.2.5 C).

In addition, the present invention is also useful for providing subfreezing temperatures liquid aqueous environments for organic and water based chemistry. As such, the present invention can facilitate desirable chemical reactions, such as enzymatic reactions in an solution in a liquid form at subzero centigrade temperatures.

In addition, the present invention is also useful in developing refrigeration systems at subzero centigrade based on liquid water solutions as the working substance.

### BRIEF DESCRIPTION OF THE DRAWINGS

FIG. 1 is an illustration of heterogeneous nucleation of ice.

FIG. 2 is a diagram of ice nucleation in an isochoric (constant volume) chamber. The subscripts o, l, and i represent the initial state of the system at the onset of freezing, liquid water, and ice, respectively. The quality is x.

FIG. 3 is a graph of pressure in an isochoric chamber as the proportion of ice (ice-I) increases.

FIG. 4. is a calculation of the critical radius of an ice-I nucleus as a function of the temperature, under isochoric and isobaric conditions. The critical radius is given in meters [m] and the temperature is in ° C.

FIG. 5. is an illustration of an isochoric cryopreservation chamber with pressure monitoring in accordance with the present invention. The system comprises a constant volume chamber that is hermetically sealed and in which the pressure is monitored with a pressure gage. The chamber is filled with fluid and is cooled by immersion in a controlled temperature bath.

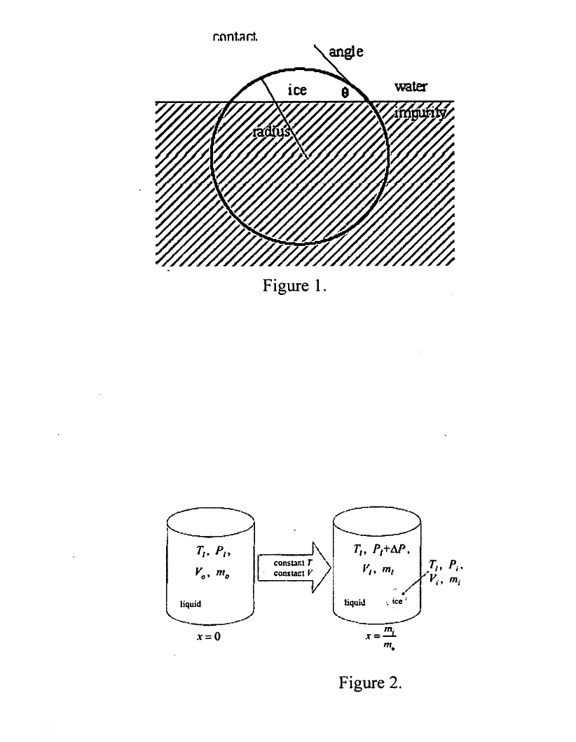

### DETAILED DESCRIPTION OF DRAWINGS

(1) Isochoric Supercooling

(a) Formation of ice in an Isochoric System

Ice-I is the ice morphology that forms at atmospheric pressure and other relatively low pressures (to about 200 MPa). Ice-I is less dense than liquid water. In accordance with the present invention, a system is provided to maintain the liquid in a constant volume (isochoric) state. Because ice-I is less dense than water, growth of ice-I in a fixed-volume chamber will cause a pressure increase. The energy required to overcome this additional pressure makes ice nucleation in the system of the present invention less thermodynamically favorable than in a comparable system that is isobaric, under any condition.

The formation of ice is a probabilistic event. In order for freezing to occur, water molecules must assemble, by means of their random movement, into an ice-like structure larger than a critical size. This critical size corresponds to an energy barrier: once this barrier is crossed, it becomes energetically favorable for more water molecules to join the ice structure, leading to the spontaneous propagation of ice. A larger critical size corresponds to a larger energy barrier. For example, under atmospheric conditions, the critical cluster size required for freezing at −5° C. is about five times greater than the critical cluster size required at −30° C., indicating that ice formation at −30° C. is highly probable.

In accordance with the present invention, the objective of the analysis is to compare the change in free energy upon the formation of an ice crystal of radius, r, and volume,

$$\frac{4 π {r}^{3}}{3} ,$$

in an isochoric system and in an isobaric system of volume V, pressure P and temperature, T.

(b) Work of Critical Cluster Formation in Isochoric and Isobaric Systems:

Ice-I, has a substantially different specific volume than water, and therefore, formation of the ice crystal in an isobaric system will cause an increase in the volume of the system, and in an isochoric system, it will cause an increase in the pressure of the system. FIG. 2 is an illustration showing the formation of a critical nucleus in an isochoric system.

In this analysis we make the following assumptions: (a) in the presence of an ice nucleus, the volume of liquid in the system is essentially equal to the total volume of the system (that is, the quality is <<1), and (b) the thermodynamic properties of liquid and ice are unchanged before and after the formation of the ice crystal.

In the case of an isobaric system, the change in free energy upon formation of an ice crystal is the sum of three components: the change in Gibbs free energy between the ice and liquid states of the molecules in the ice crystal, the energy associated with formation of the interface between ice and liquid, and the increase in volume of the system against the pressure, P. This is given by:

$$Δ G = \frac{4 π {r}^{3}}{3 {v}_{i}} ( {g}_{i} - {g}_{l} ) + 4 π {r}^{3} σ + \frac{4 π {r}^{3}}{3 {v}_{i}} ( {v}_{i} - {v}_{l} ) P$$

(gᵢ and gₗ are the specific Gibbs free energy of the ice and liquid, per unit mass; vᵢ and vₗ are the specific volumes of ice and liquid water; σ is the surface tension between ice and liquid.)

In the case of an isochoric system, the change in free energy upon formation of an ice crystal is the sum of three components: the change in Helmholtz free energy between the ice and liquid states of the molecules in the ice crystal, the energy associated with the formation of the interface between ice and liquid, and the energy associated with the pressure increase in the volume, V.

$$Δ F = \frac{4 π {r}^{3}}{3 {v}_{i}} ( {f}_{i} - {f}_{l} ) + 4 π {r}^{3} σ + V Δ P$$

(fᵢ and fₗ are the specific Helmholtz free energy of the ice and liquid, per unit mass.)

In classical nucleation analysis, the third term in the equation for the change in Gibbs free energy is assumed negligible relative to the other two terms. In contrast, we will show later that the third term in the equation for the Helmholtz free energy is not negligible with respect to the other two terms.

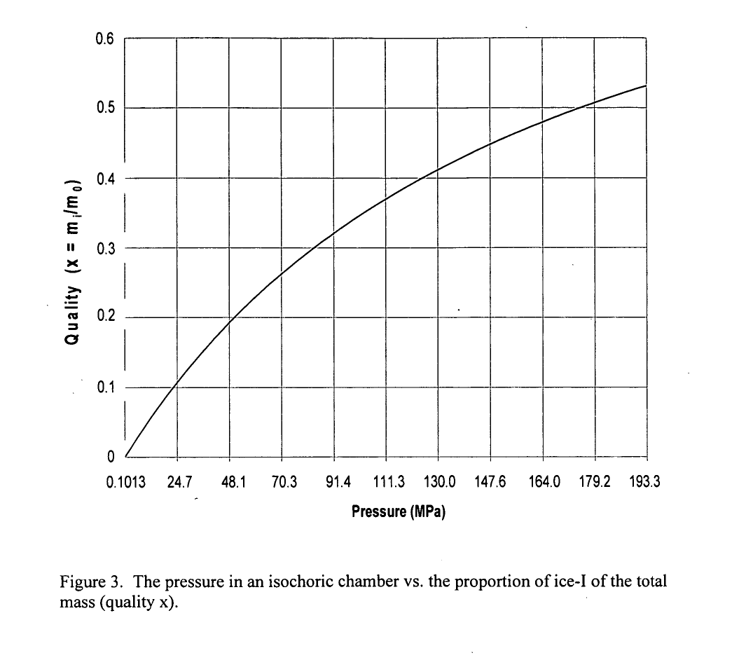

FIG. 3 shows the relationship between quality and the pressure, derived from (Rubinsky B 2005, supra) for freezing in an isochoric system. As can be seen, the pressure in an isochoric chamber increases as the proportion of ice-I of the total mass (quality x) increases.

The slope at any point on the curve given in FIG. 3 is:

$$k = \frac{ⅆ x}{ⅆ P}$$

Therefore, the last term in the equation for the Helmholtz free energy is given by:

$$V Δ P = V \frac{x}{k} = V \frac{{m}_{i}}{{m}_{l}} \frac{l}{k} = \frac{{V}_{i}}{{v}_{i}} \frac{{v}_{l}}{k} = \frac{4 π {r}^{3}}{3 {v}_{i}} \frac{{v}_{l}}{k}$$

Lastly, the change in free energy upon phase transformation is only a function of temperature and therefore:

$${g}_{i} - {g}_{l} = {f}_{i} - {f}_{l} = \frac{- L ( T )}{T} ( {T}_{m} - T )$$

(L is the latent heat of fusion per unit mass, and Tₘ is the melting temperature at a given pressure.)

The critical size for ice propagation corresponds to the maximum value of ΔG (under isobaric conditions) or ΔF (under isochoric conditions). The critical cluster radius, r_{critical}, is obtained by differentiating the ΔG and ΔF equations with respect to r and setting the result equal to zero.

For an isobaric system:

$${r}_{{critical}_{isobaric}} = \frac{- 2 σ {v}_{i}}{\frac{- L ( T )}{T} ( {T}_{m} - T ) + ( {v}_{i} - {v}_{l} ) P}$$

For an isochoric system:

$${r}_{{critical}_{isochoric}} = \frac{- 2 σ {v}_{i}}{\frac{- L ( T )}{T} ( {T}_{m} - T ) + \frac{{v}_{l}}{k}}$$

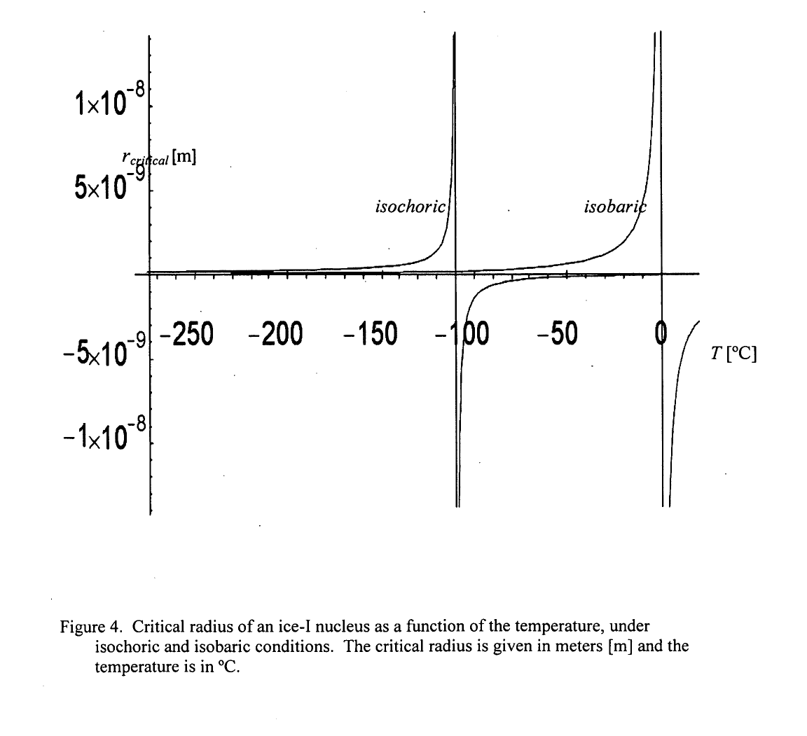

Substituting the data from Table 1 (Rubinsky B, P. P., Carlson M E (2005). “The thermodynamic principles of isochoric cryopreservation.” Cryobiology 50: 121-138.) in the analysis reveals important differences in the critical radius for water under isochoric and isobaric conditions, which are illustrated in FIG. 4.

(c) Summary of Analysis/Experimental Results

FIG. 4 shows that in an isobaric system, the critical radius is asymptotic at T=0° C., because an infinitely large ice cluster is required for homogeneous nucleation at that temperature. The formation of ice becomes more favorable as T decreases, and consequently, a smaller critical cluster is required at lower temperatures.

In the isochoric system, the critical radius is asymptotic at T=−109° C. No finite, positive values of the critical radius exist until T<−109° C. At temperatures below −109° C., the isochoric system behaves in a manner that is similar to the isobaric system. Thus, in theory and for an ideal system, homogeneous nucleation is not possible under isochoric conditions until the system has been subcooled below −109° C.

The real values for isochoric homogeneous nucleation in a biological system may be higher. Biological tissues have additional components to water, which may have a higher compressibility than water. If a compressible gas is included in the system, the value of k would presumably increase, and the term

$$\frac{{v}_{l}}{k}$$

would have a smaller effect on the energy of cluster formation and critical radius. Furthermore, nucleation may be heterogeneous. Nevertheless, several important concepts emerge from these calculations. First, isochoric subcooling of water depresses the probable formation of a critical ice nucleus to substantially lower temperatures than isobaric cooling. This shows the potential to promote supercooling and vitrification. A second result is that these calculations are independent of the cooling rate. Unlike current cryopreservation protocols, which require fast cooling rates in order to reduce the probability of ice nucleation, isochoric cooling can take place at any rate. Thus, in the region of temperatures in which isochoric cooling affects nucleation, the cooling pathway can be optimized for cell type or available technology. Furthermore, in an isochoric system, the pressure will not change until ice nucleation occurs. Therefore, a system initially at atmospheric pressure will remain at atmospheric pressure while being subcooled to the nucleation temperature. Therefore, biological substances could be stored in an isochoric system at low temperatures without freezing and without pressure.

The same calculations indicate that isochoric cooling will also depress heterogeneous nucleation (including intracellular and intramatrix sites), although measures to avoid heterogeneous nucleation may be beneficial during cryopreservation. In accordance with aspects of the present invention, such measures include, but are not limited to, eliminating impurities, ensuring that water-contacting surfaces are hydrophobic and scratch-free, or applying anti-nucleating agents (such as an oily hydrocarbon coating or antifreeze proteins) to the surfaces of biological substances. In accordance with the present invention, combining isochoric cooling with cryoprotectants or other vitrification solutions will advance cryopreservation by utilizing the advantages of both of these ice avoidance techniques.

The same calculations also indicate that isochoric systems will also inhibit the formation of an ice crystal during warming and inhibit recrystallization.

The present system advantageously leads to a reduction in the probability for nucleation in water and aqueous solutions. The present invention thus has applications in: (a) preservation of biological materials in a liquid form in a thermodynamically supercooled state, (b) inhibition of ice formation inside cells (the system is the space inside cells) during rapid cooling and (c) vitrification. As explained, the present system provides a system of cryopreservation in an isochoric chamber. This system advantageously leads to vitrification and eliminates or reduces the need for elevated pressures, high concentrations of chemical additives and high cooling and warming rates in the current techniques.

The present invention is thus particularly useful for preserving biological materials including, but not limited to: solutions of biological compounds, cell components, cells, tissues organs, and organisms at subzero centigrade temperatures. Reducing the temperature has the effect of reducing the rate of chemical reactions and can be used for the preservation of these compounds. In preferred aspects, the reduced temperature range is from zero centigrade to absolute zero (−273.25 C).

In optional aspects of the invention, the present system can be used to provide subfreezing temperatures liquid aqueous environments for organic and water based chemistry. As such, the present invention can facilitate desirable chemical reactions, such as enzymatic reactions in a solution in a liquid form at subzero centigrade temperatures.

In addition, the present invention is also useful in developing refrigeration systems at subzero centigrade based on liquid water solutions as the working substance.

(2) An Exemplary System of Cryopreservation in Accordance with the Present Invention

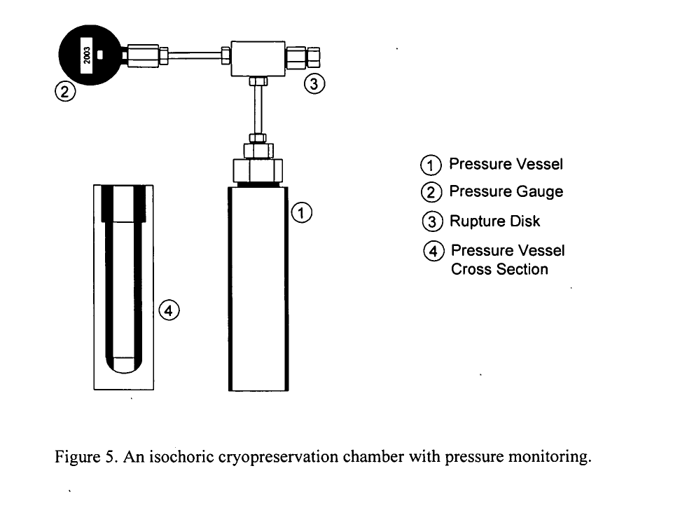

FIG. 5 illustrates an exemplary system for isochoric cryopreservation in accordance with the present invention. The system comprises a constant volume chamber in pressure vessel 1, a pressure gauge 2 and a rupture disk 3. Note: the constant volume chamber in pressure vessel 1 is seen in the cross sectional view labeled 4 in the Fig. The constant volume chamber in pressure vessel 1 is preferably hermetically sealed, and the pressure therein is monitored with pressure gage 2. Optionally, constant volume chamber in pressure vessel 1 can be made of stainless steel, but the present invention is not so limited. The chamber in pressure vessel 1 is filled with fluid and is cooled by immersion in a controlled temperature bath (not shown). If ice nucleates in the chamber, the pressure of the system increases and the nucleation can be detected by the pressure gage.

In accordance with the present invention, cryopreservation may be achieved as follows:

First, a tissue or organ or other biological material is placed in the preserving fluid in the chamber. Second, the chamber is sealed with care to completely fill the chamber with fluid. Third, the chamber is cooled with an external cooling source while the volume of the chamber is kept constant (isochoric). This cooling can be optionally achieved by immersing the chamber in a controlled temperature bath. According to the present invention, the fluid of interest for the biological material will be supercooled yet remain in a liquid state.

The fluid of interest in the system can be physiological saline solutions or hypothermic preservation solutions or physiological solutions with cryoprotectants according to the cryopreservation protocol of interest. The preservation temperature can be determined according to the cryopreservation protocol of interest. The cooling and warming of the chamber with the biological materials can be designed to obtain the cryopreservation protocol of interest. With all these various applications, the constant volume chamber reduces the probability of ice nucleation (relative to a similar chamber that is not isochoric but rather isobaric) and obtain the benefits of the reduction in probability for ice nucleation. The present invention can be used for preservation in a supercooled state, preservation with freezing to reduce the probability of formation of intracellular ice and preservation with vitrification to reduce the probability of intracellular ice formation during cooling and warming.

In one aspect of the invention, heterogeneous nucleation can be further avoided by eliminating impurities and insuring that all surfaces in contact with the fluid are hydrophobically coated and scratch free. In one optional aspect, heterogeneous nucleation on the organ surface can be prevented by first coating the organ with an oily hydrocarbon or using such compounds as antifreeze proteins.

In optional aspects, a compound with cryoprotective properties, or properties that promote vitrification may be added to the fluid, or to the biological sample. Such compounds may include glycerol, ethylene glycol, and dimethyl sulfoxide (DMSO).

| | |
|---|---|
| σ = (28.0 + 0.25T)10⁻³ J/m² | 0 ≧ T ≧ −36° C. |
| σ = 0.0190 J/m² | −36° C. ≧ T |
| ${v}_{i} = {[ {\sum }_{n = 0}^{2} {a}_{n} {T}^{n} ]}^{- 1} \times {10}^{- 3} {m}^{3} / kg$ | 0 ≧ T ≧ −180° C. |
| ${v}_{l} = {[ {\sum }_{n = 0}^{6} {a}_{n} {T}^{n} ]}^{- 1} \times {10}^{- 3} {m}^{3} / kg$ | 0 ≧ T ≧ −33° C. |
| ${v}_{l} = {[ {\sum }_{n = 0}^{6} {{a}_{n} ( T - 4 )}^{n} ]}^{- 1} \times {10}^{- 3} {m}^{3} / kg$ | −33° C. > T > −45° C. |
| vₗ = vᵢ m³/kg | −45° C. ≧ T ≧ |
| | −180° C. |
| k = 5.4 × 10⁻⁹ m³/J | x << 1 |
| L(T) = (6.95T + 4264.8)(1/18.015) × 10³ J/m³ | T in degrees K. |
| P = 101300 N/m² | (atomospheric pressure) |
| Tₘ = 273 K. | (melting temperature of |
| | ice at atmospheric |
| | pressure) |

TABLE 1
Data in the analysis from Rubinsky B, P. P., Carlson ME (2005). “The
thermodynamic principles of isochoric cryopreservation.”
Cryobiology 50: 121-138.

parameters for νᵢ
a₀ = 0.9167
a₁ = −1.75 × 10⁻⁴
a₂ = −5.0 × 10⁻⁷
parameters for νₗ
a₀ = 0.99986
a₁ = 6.690 × 10⁻⁵
a₂ = −8.486 × 10⁻⁶
a₃ = 1.518 × 10⁻⁷
a₄ = −6.9984 × 10⁻⁹
a₅ = −3.6449 × 10⁻¹⁰
a₆ = −7.497 × 10⁻¹²

What is claimed:

1. A method of cryopreservation of a biological sample, comprising:
placing a biological sample in a fluid in a chamber; and
supercooling the fluid in the chamber under isochoric conditions, without actively inducing ice nucleation in the fluid, thereby cryopreserving the biological sample.

2. The method of claim 1, wherein the fluid is an aqueous solution.

3. The method of claim 1, wherein the biological sample is selected from the group consisting of a cell, a group of cells, an organ and an organism.

4. The method of claim 1, further comprising:
adding a compound with cryoprotective properties to the fluid.

5. The method of claim 4, wherein the compound with cryoprotective properties is selected from the group consisting of glycerol, ethylene glycol, and DMSO.

6. The method of claim 1, further comprising:
adding a chemical that promotes vitrification of the fluid.

7. The method of claim 6, wherein the chemical that promotes vitrification to the fluid is selected from the group consisting of glycerol, ethyle glycols and DMSO.

8. The method of claim 1, further comprising:
adding a chemical that inhibits nucleation in the fluid.

9. The method of claim 8, wherein the chemical that inhibits nucleation in the fluid is selected from the group consisting of antifreeze proteins and oily hydrocarbons.

10. The method of claim 1, wherein the biological sample comprises a compound with cryoprotective properties.

11. The method of claim 10, wherein the compound with cryoprotective properties is selected from the group consisting of glycerol, ethylene glycol, and DMSO.

12. The method of claim 1, wherein the biological sample comprises a chemical that promotes vitrification of the biological sample.

13. The method of claim 12, wherein the chemical that promotes vitrification of the biological sample is selected from the group consisting of glycerol, ethylene glycols and DMSO.

14. The method of claim 1, wherein the fluid is supercooled to temperatures in the range from 0 C to −273.25 C.

15. The method of claim 1, wherein the fluid is supercooled by immersing the chamber in an exterior fluid.

16. The method of claim 1, wherein the chamber does not contain ice nucleating agents.

17. The method of claim 1, wherein the chamber does not contain gases.

18. The method of claim 1, wherein the chamber does contain materials that absorb gases.

19. The method of claim 1, wherein the chamber contains agents that inhibit nucleation.

20. The method of claim 19, wherein the agents that inhibit nucleation are selected from the group consisting of antifreeze proteins and thermal histeresys proteins.

21. A method of cryopreservation of a biological sample, comprising:
placing a biological sample in a fluid in a chamber; and
supercooling the fluid in the chamber under isochoric conditions, thereby reducing the probability of ice nucleation in the fluid, thereby improving the probability for cryopreserving the biological sample.

22. The method of claim 21, wherein the fluid is an aqueous solution.

23. The method of claim 21, wherein the biological sample is selected from the group consisting of a cell, a group of cells, an organ and an organism.

24. The method of claim 21, further comprising:
adding a compound with cryoprotective properties to the fluid or to the biological sample.

25. The method of claim 24, wherein the compound with cryoprotective properties is selected from the group consisting of glycerol, ethylene glycol, and DMSO.

26. The method of claim 21, further comprising:
adding a chemical that promotes vitrification of the fluid or the biological sample.

27. The method of claim 26, wherein the chemical that promotes vitrification is selected from the group consisting of glycerol, ethyle glycols and DMSO.

28. The method of claim 21, further comprising:
adding a chemical that inhibits nucleation in the fluid.

29. The method of claim 28, wherein the chemical that inhibits nucleation in the fluid is selected from the group consisting of antifreeze proteins and oily hydrocarbons.

30. A system for cryopreservation of a biological sample, comprising:
an isochoric chamber; and
a supercooling system adapted to cool contents of the isochoric chamber to temperatures in the range of from 0 C to −273.25 C.

31. The system of claim 30, wherein the supercooling system is a fluid bath in which the isochoric chamber is immersed.

32. The system of claim 30, wherein the isochoric chamber is hermetically sealed.

33. The system of claim 30, further comprising:
a system for monitoring the pressure in the isochoric chamber

34. The system of claim 30, further comprising:
a fluid in the isochoric chamber; and
a biological sample in the fluid.

35. The system of claim 30, wherein the chamber does not contain ice nucleating agents.

36. The system of claim 31, wherein the chamber does not contain gases.

37. The system of claim 31, wherein the chamber contains materials that absorb gases.

38. The system of claim 31, wherein the chamber contains agents that inhibit nucleation.
